(19) United States
(12) Patent Application Publication
(10) Pub. No.: US 2008/0044504 A1
(43) Pub. Date: Feb. 21, 2008

(54) Pomegranate Juice, Pomegranate Juice Powder and Process for Producing the Powder

(75) Inventors: Eriko Komaki, Kyoto (JP); Isafumi Maru, Kyoto (JP); Yasuhiro Ohta, Kyoto (JP)

Correspondence Address: FITCH, EVEN, TABIN & FLANNERY, P. O. BOX 18415, WASHINGTON, DC, 20036, US

(73) Assignee: Marukin Bio, Inc., Uji-shi (JP)

(21) Appl. No.: 11/631,471

(22) PCT Filed: Jul. 5, 2005
(86) PCT No.: PCT/JP05/12380

§ 371 Date: Jan. 4, 2007

Related U.S. Application Data
(60) Provisional application No. 60/590,401, filed on Jul. 22, 2004.

(30) Foreign Application Priority Data
Jul. 5, 2004 (JP) 2004-198558

(51) Int. Cl. A61K 36/00; A23L 2/02; C12G 1/022; A23L 2/08

(52) U.S. Cl. 424/769; 426/15; 426/471; 426/599

(57) ABSTRACT

A pomegranate juice with enhanced estrogen-like activity per unit amount, a pomegranate juice powder, a process of producing the powder, and foods, feeds, pharmaceutical compositions and the like containing the powder. A pomegranate juice containing substantially no glucose and no fructose; a pomegranate juice containing not more than about 50% of the glucose and fructose of a pomegranate juice and having about 50% or more of the estrogen-like activity of the pomegranate juice; a pomegranate juice having an estrogen-like activity of about 96 U per gram of glucose and fructose contained in the juice; a pomegranate juice powder having an estrogen-like activity of about 40 U or more per gram of the powder; a pomegranate juice powder containing glucose and/or fructose and having an estrogen-like activity of about 80 U or more per gram of glucose and fructose contained therein; and like powders.

### TECHNICAL FIELD

The present invention relates to a pomegranate juice with enhanced estrogen-like activity per unit amount, a pomegranate juice powder, a process for producing the powder, and foods, feeds, pharmaceutical compositions and the like containing the powder.

### BACKGROUND ART

Substances having estrogen-like activity, derived from plant-based foods, are called phytoestrogens. Foods containing phytoestrogens are considered to be effective in preventing osteoporosis and hyperlipidemia associated with menopausal disorders, preventing obesity, etc., and preventing or treating breast cancer and uterine cancer.

Isoflavones (daidzein, genistein, etc.) that are structurally similar to estrogen are contained in soybeans. Diethylstilbestrol (hereinafter sometimes referred to as “DES”) is known as a synthetic estrogen. In comparing daidzein, genistein and DES in terms of their affinity to an estrogen receptor, DES has the highest affinity. It has been reported that when the binding rate of DES to the estrogen receptor is defined as 100%, the binding of estrogen is 42%, the binding of daidzein is 0.04% and the binding of genistein is 1.7% (see Non-patent Document 1).

Pomegranate juice is known to have estrogen-like activity. In old times, the presence of estrone (molecular weight: 270.4) and coumesterol (molecular weight: 268.2) as substances having estrogen-like activity in pomegranate juice was reported (see Non-patent Documents 2, 3 and 4). With the expectation of such estrogen-like activity effects, pomegranate juice started being drunk. However, later, there was a report denying the presence of estrogen in pomegranate juice (see Non-patent Document 5). However, recently, it has been reconfirmed that pomegranate juice clearly has estrogen-like activity (see Non-patent Document 6), and pomegranate juice has started being drunk again. However, the component(s) having estrogen-like activity have yet to be specified.

Pomegranate juice generally traded as a food material is a 5-fold concentrated juice with a Brix of about 65°, obtained by squeezing the fruit, followed by concentration. Commercially available pomegranate juice beverages include 5-fold concentrated juices, straight juices, and mixtures with other fruits. When a 5-fold concentrated juice is drunk with the expectation of estrogen-like activity, one needs to ingest at least 30 ml of the juice daily. However, since concentrated juice is a liquid, the juice is unsuitable for transportation and is not easy to ingest anywhere other than at home and furthermore, it needs to be stored in a refrigerator. Therefore, it has been desired to form pomegranate juice into solids such as pills and tablets advantageous in terms of portability, storage, etc., while retaining the estrogen-like activity. However, when pomegranate juice is merely dried, the juice becomes syrupy and cannot be pulverized. This is attributable to the hygroscopicity of saccharides (in particular, glucose and fructose) contained in large amounts in pomegranate juice.

Generally, fruit juice powders are often used by being dissolved in water to reconstitute a fruit juice or by being added to foods, etc. to add the taste, flavor, nutritive value, etc. of the original fruit. Therefore, when a fruit juice is pulverized, pulverization methods minimizing the loss of components contained in the fruit juice (in particular, components affecting the taste and flavor) are preferably used. However, as mentioned above, when pomegranate juice is merely dried, it becomes syrupy and cannot be pulverized. Therefore, pulverization methods comprising adding a drying aid such as dextrin, lactose or the like to a pomegranate juice so as to suppress glucose and fructose from absorbing water, followed by pulverizing the resulting juice by spray-drying or lyophilization have been used. However, since the amount of drying aid that needs to be added for the pulverization is usually from 1 to less than 20 times the saccharide content of the original fruit juice (by weight) (e.g., about twice or more when dextrin is used as a drying aid in the pulverization of pomegranate juice), a powder pulverized by this method has a high saccharide content per unit weight. Therefore, when powder obtained by this method is ingested to intake the same amount of fruit juice-derived components as is conventionally ingested by drinking a fruit juice, there arises a problem that a large amount of saccharides (high calorie substances) are ingested at the same time. For example, while calorie intake by ingesting 30 ml of concentrated pomegranate juice is about 70.8 kcal., calorie intake by ingesting a powder obtained by pulverizing the same amount of concentrated pomegranate juice using dextrin as a drying aid is about 175.5 kcal. This example is a caloric comparison in the intake of the minimum amount of a concentrated pomegranate juice expected to exhibit estrogen-like activity. When a larger amount of juice or powder is ingested, calorie intake increases. Thus, since intake of fruit juice powder using a drying aid results in high calorie intake and menopausal women are particularly prone to developing obesity and hyperlipidemia, intake of such a high-calorie powder has problems.

Therefore, methods for pulverizing fruit juice without needing to use a drying aid or with reduced use of a drying aid have been studied. Examples of such methods include techniques described in the following patent publications:
A method comprising mixing a fruit juice and a proanthocyanidin-containing composition, followed by pulverizing the mixture (Patent Document 1).
A method of producing an orange juice powder comprising adding 5 to 30 wt. % of an inactive solid mainly consisting of pulp and the like obtained by isolation from orange juice and homogenizing the mixture, followed by centrifugal spray drying (Patent Document 2).
A method of producing a fruit juice powder comprising storing orange juice in a frozen state at −10° C. for 1 week or more, then thawing the juice so as to separate pulp from the juice, removing the pulp by a centrifuge, followed by spray drying (Patent Document 3).
A method of producing a fruit juice powder comprising adding pectin to a raw fruit juice, adjusting the pH to 2.8 to 8.4 and heating, followed by spray drying at a low outlet air temperature of 50° C. to 70° C. (Patent Document 4).
A method of producing a natural fruit juice-containing powder comprising mixing a natural fruit juice with a water adsorptive or anhydrous solid natural sweetener to form a solid containing the natural fruit juice, followed by pulverizing the solid (Patent Document 5).

[Non-patent Document 1]
Endocrinology, 139, 4232 (1998) KUIPER, G. G. J. M. et al.

[Non-patent Document 2]
J. Chromatogr., 438, 438 (1988) MONEAM, N. M. A.; SHARAKY, A. S. E. and BARDERELDIN, M. M.

[Non-patent Document 3]
Naturwissenschaften, 52, 451 (1965) HEFTMAN, E.; KO, S. T. and BENNET, R. D.

[Non-patent Document 4]
Phytochemistry, 10, 2215 (1971) DEAN, P. O. G.; EXLEY, D. and GOODWIN, T. W.

[Non-patent Document 5]
Food Science Journal, No. 268, 102-110 (2000), National Consumer Affairs Center of Japan

[Non-patent Document 6]
Journal of the Japanese Society for Food Science and Technology, Vol. 48, No. 2, 146-149 (2001), Maru, Onishi, Yamaguchi et al.

[Patent Document 1]
Japanese Unexamined Patent Publication No. 2001-274604

[Patent Document 2]
Japanese Examined Patent Publication No. 1982-7705

[Patent Document 3]
Japanese Examined Patent Publication No. 1977-14302

[Patent Document 4]
Japanese Unexamined Patent Publication No. 1979-129154

[Patent Document 5]
Japanese Unexamined Patent Publication No. 1978-24054

### DISCLOSURE OF THE INVENTION

An object of the invention is to provide a pomegranate juice with enhanced estrogen-like activity per unit amount, a pomegranate juice powder, a process for producing the powder, and foods, feeds, pharmaceutical compositions and the like containing the powder.

As described above, a substance with estrogen-like activity in pomegranate juice is expected to be a low molecular weight compound, such as estrogen analogues. Therefore, although removing a lower molecular weight fraction from pomegranate juice is expected to cause the loss of estrogen-like activity, the present inventors have unexpectedly found that estrogen-like activity is retained. The inventors produced a pomegranate juice powder with enhanced estrogen-like activity per unit amount by removing saccharides from pomegranate juice so as to reduce the amount of a drying aid used, while retaining estrogen-like activity, and thereby accomplished the present invention.

Thus the present invention provides the following pomegranate juices, pomegranate juice powders, processes for producing the powders, and foods and feeds containing the powders.
Item 1. A fruit juice derived from pomegranate juice, comprising substantially no glucose and no fructose.
Item 2. A fruit juice derived from a pomegranate juice, the fruit juice containing not more than 50% of the glucose and fructose of the pomegranate juice and having about 50% or more of the estrogen-like activity of the pomegranate juice.
Item 3. A fruit juice derived from pomegranate juice and having an estrogen-like activity of about 96 U or more per gram of glucose and fructose contained in the fruit juice.
Item 4. A pomegranate juice powder having an estrogen-like activity of about 40 U or more per gram of the powder.
Item 5. The powder according to item 4 having an estrogen-like activity of about 40 to about 20,000 U per gram of the powder.
Item 6. The powder according to item 4 or 5 further comprising a drying aid.
Item 7. A pomegranate juice powder containing glucose and/or fructose and having an estrogen-like activity of about 80 U or more per gram of the glucose and fructose contained therein.
Item 8. The powder according to item 7 further comprising a drying aid.
Item 9. A process for producing a pomegranate juice powder comprising a saccharide removal step of removing glucose and fructose from a pomegranate juice, and a drying step of drying the juice obtained by the saccharide removal step.
Item 10. The process according to item 9 wherein the saccharide removal step is performed by a membrane treatment using at least one membrane with a molecular weight cut-off of about 200 to about 100,000 selected from the group consisting of dialysis membranes, ultrafiltration membranes, nano filters, and reverse osmotic membranes, or by chromatography using a packing with a molecular weight cut-off of about 200 to about 100,000 to obtain a higher molecular weight fraction.
Item 11. The process according to item 9 wherein the saccharide removal step is performed by a microbiological method comprising treating the juice with a microorganism which utilizes glucose and/or fructose to produce an alcohol, or by an enzymatic method comprising treating the juice with an enzyme that uses glucose and/or fructose as substrate(s).
Item 12. The process according to item 10 or 12 which comprises an additional step, between the saccharide removal step and the drying step, of adding a drying aid to the juice from which saccharide(s) have been removed.
Item 13. A pomegranate juice powder obtainable by the method of any one of items 9 to 12 and having estrogen-like activity.
Item 14. A food comprising the pomegranate juice powder of any one of items 4 to 8 and 13.
Item 15. A feed comprising the pomegranate juice powder of any one of items 4 to 8 and 13.
Item 16. A pharmaceutical composition comprising an effective amount of the pomegranate juice powder of any one of items 4 to 8 and 13.
Item 17. The pharmaceutical composition according to item 16 which is a preventive or therapeutic agent for a disease associated with female hormone reduction.
Item 18. A method of preventing or treating a disease associated with a female hormone, comprising orally administering an effective amount of the pomegranate juice powder of any one of items 4 to 8 and 13 to a subject.

In the invention, estrogen-like activity is determined from a comparison of between the IC₅₀ of a sample and the IC₅₀ of diethylstilbestrol (hereinafter sometimes referred to as “DES”) obtained by using competitive binding reactions with an estrogen receptor, with the activity of 1 μg of DES being defined as 1 U (unit). More specifically, the activity was determined in the following manner. First, 17β-estradiol and a DES solution are supplied in the presence of estrogen receptor α and subjected to competitive binding. Free 17β-estradiol not bound to the receptor is measured by an enzymatic immunoassay using a 17β-estradiol antibody to determine the binding ratios of 17β-estradiol and DES to the receptor. The concentration (IC₅₀) of DES required for 50% inhibition of the binding of 17β estradiol is calculated from the measurement results. When the IC₅₀ of DES is 2.5 ng/ml, the estrogen-like activity of 1 ml of this DES solution is 0.0025 U since the activity of 1 μg of DES is defined as 1 U.

A sample solution (for example, a fruit juice) is then used in place of the aqueous DES solution to determine the 50% inhibitory concentration (IC₅₀) of the sample in a similar manner. When the IC₅₀ of the fruit juice is 80 μg/ml, the activity of 1 g of the fruit juice is equivalent to the activity (0.0025 U) of 1 ml (2.5 ng/ml) of DES sample. The specific activity is calculated as 1/32000, from 2.5 ng/80 μg. Since the activity per gram of DES is 1×10⁶ U (1 U/μg×10⁶), the activity of 1 g of the fruit juice is 31.25 U (1×10⁶ U× 1/32000).

Furthermore, when 1 ml of this fruit juice contains 0.65 g of saccharides, the estrogen-like activity per gram of saccharide is expressed as 48 U (i.e., 31.25 U×1/0.65).

Although a representative example of pomegranate to be used in the invention is Punica granatum, a pomegrante belonging to the family of Punicaceae, usable pomegranates are not limited thereto. Examples of commonly used pomegranate juices include straight juices obtained by squeezing pomegranate fruit, concentrated juices obtained by concentrating such straight juices, and other pomegranate juices. Straight juices or 2 to 5 times concentrated juices are preferable.

One pomegranate juice of the invention is different from the commonly used pomegranate juices in that the juice comprises substantially no glucose and no fructose. Another pomegranate juice of the invention contains not more than 50% of the glucose and fructose of a natural pomegranate juice, and has about 50% or more of the estrogen-like activity of the natural pomegranate juice. Another pomegranate juice of the invention has an estrogen-like activity of about 96 U or more per gram of the total amount of glucose and fructose contained in the juice.

Pomegranate juice of the invention can be obtained by removing glucose and fructose from commonly used pomegranate juice. More specifically, pomegranate juice of the invention can be obtained by gel filtration or membrane treatment using dialysis membranes, ultrafiltration membranes, nano filters, or reverse osmotic membranes with a molecular weight cut-off of about 200 to about 100,000 to remove saccharides and a lower molecular weight fraction.

The drying aid usable in the invention is not particularly limited and examples thereof include edible drying aids. conventionally used in the production of known powders. The addition of a drying aid to a pomegranate juice suppresses hygroscopicity of the glucose and fructose which hinders the pulverization of pomegranate juice. According to the present invention, the addition of a drying aid becomes unnecessary or the amount of drying aid added can be reduced by removing the glucose and fructose from pomegranate juice without losing the estrogen-like activity of the pomegranate juice. Examples of usable drying aids include substances known as excipients, such as soluble starch, dextrin, malto-dextrin, lactose, powder sugar, cornstarch, and crystalline cellulose and the like. Soluble starch, dextrin, malto-dextrin and lactose are preferable.

One pomegranate juice powder of the invention has an estrogen-like activity of about 40 U or more per gram of the powder. Preferably, the pomegranate juice powder has an estrogen-like activity of about 40 U to about 20,000 U, and more preferably about 48 U to about 3000 U, per gram of the powder.

Another pomegranate juice powder according to the invention may comprise glucose and/or fructose and have an estrogen-like activity of about 80 U or more per gram of the total amount of glucose and fructose contained in the powder.

As long as the pomegranate juice powder of the invention has the above estrogen-like activity, the powder may or may not contain drying aid(s). When the glucose and fructose is completely removed from a pomegranate juice, a powder free of a dying aid can be obtained. Such a powder has a very potent estrogen-like activity per unit amount. When the glucose and fructose contained in pomegranate juice are partially removed and a drying aid is used to dry the glucose and fructose remaining in the juice, a powder containing the drying aid can be obtained. Since there is no need to completely remove the glucose and fructose, such a powder is economically advantageous. Since the powder of the invention has the above estrogen-like activity even when containing a drying aid, the powder is different from conventional pomegranate juice powders. When the pomegranate juice powder of the invention contains drying aid(s), the drying aid content is preferably about 45 wt. % or less, more preferably about 40 wt. % or less, and even more preferably about 30 wt. % or less.

Since excipients such as dextrin and lactose are usually added to produce pills, tablets, capsules and the like, the resulting products tend to have a high caloric content. Since the powder of the invention contains a reduced amount of drying aid while retaining estrogen-like activity, the powder has a low caloric content and is capable of reducing calorie intake. This is because when the caloric content derived from glucose and fructose is low, the kinds and amounts of other additives used can be more freely selected in producing a food of the same calorific value. The saccharide content of the pomegranate juice powder can be determined by enzymatic assays and HPLC methods.

The content of pomegranate juice-derived components with a molecular weight of about 200 to about 100,000 in the pomegranate juice powder of the invention is preferably about 1.6 wt. % or more, and more preferably about 50 to about 100 wt. %, of the pomegranate juice-derived components contained therein. Further preferably, the content of pomegranate juice-derived components with a molecular weight of about 500 to about 30,000 in the powder is about 55 wt. % or more, and particularly preferably about 60 to about 100 wt. %. When the content of pomegranate juice-derived components with a molecular weight of about 200 to about 100,000 is within the above-mentioned range, little loss of estrogen-like activity results. When the content of pomegranate juice-derived components with a molecular weight of about 500 to about 100,000 is within the above-mentioned range, the estrogen-like activity loss is further less. When the content of pomegranate juice-derived components with a molecular weight of about 500 to about 30,000 is within the above-mentioned range, the estrogen-like activity loss is very little.

The process of producing a pomegranate juice powder according to the invention comprises a saccharide removal step of removing glucose and fructose from a pomegranate juice, and a drying step of drying the juice obtained by the removal step.

In the invention, “removing glucose and fructose from a pomegranate juice” means partially or completely removing the glucose and fructose from a pomegranate juice to reduce or completely remove the glucose and fructose. “Saccharide removal” refers to partial or complete removal of saccharides.

In the saccharide removal step, saccharides, in particular, glucose and fructose, are removed from a pomegranate juice to achieve a desired saccharide content. Examples of methods usable in the saccharide removal step include methods of removing glucose and fructose by separating a fraction containing glucose and fructose from either a fraction not containing glucose and fructose or a fraction with a low content of glucose and fructose, according to molecular weight, to obtain a fraction containing neither glucose nor fructose (e.g., membrane treatment methods using dialysis membranes, ultrafiltration membranes, nano filters, reverse osmotic membranes and the like, gel filtration chromatography and like chromatographies); microbiological methods using microorganisms which utilize glucose and fructose; and enzymatic methods using enzymes which utilize glucose and fructose as substrates. According to such methods, glucose and fructose can be removed, while reducing loss of estrogen-like activity.

In methods using dialysis membranes, for example, a pomegranate juice is placed under atmospheric or reduced pressure in a dialysis tube with a molecular weight cut-off of about 200 to about 100,000, preferably about 500 to about 100,000, and more preferably about 500 to about 30,000, and immersed in tap water, distilled water, sterilized water, ultrapure water or the like to separate a lower molecular weight fraction from a higher molecular weight fraction to obtain the higher molecular fraction, thus giving a pomegranate juice from which glucose and fructose has been removed.

In methods using ultrafiltration membranes, nano filters, or reverse osmotic membranes, a pomegranate juice is subjected to a membrane treatment according to a conventional method to separate a lower molecular weight fraction from a higher molecular weight fraction to obtain the higher molecular fraction, thus giving a pomegranate juice from which glucose and fructose has been removed.

The chromatography is not particularly limited and any chromatography that is capable of separating glucose and fructose from a pomegranate juice based on molecular weight difference can be used, and known chromatographies are broadly usable. For example, according to gel filtration methods, a pomegranate juice is added to a column packed with a gel with a molecular weight cut-off of about 200 to 100,000, preferably about 500 to about 100,000, and more preferably about 500 to about 30,000, followed by eluting with water or an appropriate buffer such as a phosphate buffer to separate a lower molecular fraction from a higher molecular fraction to obtain the higher molecular fraction, thus giving a pomegranate juice from which glucose and fructose have been removed. Examples of gels usable in gel filtration include Sephadex G-15 (molecular weight cut-off: 1500 or less), Sephadex G-25 (molecular weight cut-off: 1000 to 5000), Sephadex G-50 (molecular weight cut-off: 1500 to 30000), Sephadex G-75 (molecular weight cut-off: 3000 to 80000), Sephadex G-100 (molecular weight cut-off: 4000 to 150000), Sephadex G-150 (molecular weight cut-off: 5000 to 300000), Sephadex G-200 (molecular weight cut-off: 5000 to 600000) and the like.

In microbiological methods using microorganisms which utilize glucose and fructose, usable microorganisms are, for example, Saccharomyces cereviciae, Zygosaccharomyces rouxii and like yeasts, Streptococcus lactis, S. faecalis, S. thermophilus, Lactobacillus bulgaricus, Lac. lactis, Lac. acidophilus and like lactic acid bacteria, Aspergillus oryzae, Aspergillus niger and like fungi, and like microorganisms. Yeasts are preferable. By adding a microorganism to a pomegranate juice, followed by culturing under culture conditions suitable for the microorganism, glucose and fructose in the pomegranate juice can be converted to an alcohol by the action of the microorganism to give a pomegranate juice from which glucose and fructose have been removed. When Saccharomyces cereviciae is used, for example, Saccharomyces cereviciae can be usually inoculated in a diluted juice prepared by diluting a 5 times concentrated pomegranate juice at least twice, preferably about 5 to 30 times, followed by stationary culturing usually at about 4° C. to about 60° C., and preferably at about 20° C. to about 40° C., usually for 1 day or more, and preferably for about 5 to about 10 days to give a pomegranate juice from which glucose and fructose have been removed. In the case that the fruit alcohol is problematic for persons underage or persons having a low tolerance to alcohol, the residual alcohol can be removed by distillation, membrane filtration or acetic acid fermentation.

In enzymatic methods using enzymes which utilize glucose and fructose as substrates to remove glucose and fructose from a pomegranate juice, usable enzymes are, for example, glucose dehydrogenase, glucose oxidase, fructose dehydrogenase and like enzymes. By adding an enzyme to a pomegranate juice and maintaining the resulting juice under enzymatic reaction conditions suitable for the enzyme, glucose and fructose in the pomegranate juice can be converted to other substances to give a pomegranate juice from which glucose and fructose have been removed. Since cell lysates obtained by culturing microorganisms that produce such enzymes, followed by disruption, contain such useful enzymes, such lysates can be used in a manner similar to enzymes. When glucose oxidase or fructose dehydrogenase is used, the enzyme is usually used in a proportion of about 1 to about 2000 units, and preferably about 100 to about 1000 units, per ml of a 5 times diluted pomegranate juice and the reaction is allowed to proceed, usually at about 10° C. to about 90° C., and preferably at about 20° C. to about 60° C., usually for about 1 minute or more, and preferably for about 10 minutes to about 60 minutes, thus giving a pomegranate juice from which glucose and fructose have been removed.

The pomegranate juice obtained in the saccharide removal step is dried for pulverization. When the amount of saccharide removal is small, the pomegranate juice is not pulverized by simply drying the juice as is. Therefore, a drying aid addition step of adding a drying aid before drying is provided.

In the drying aid addition step, a drying aid is added to the pomegranate juice obtained in the saccharide removal step. The amount of drying aid added can be appropriately selected according to the saccharide content of the pomegranate juice. The amount of drying aid is usually 0 to about 98.4 wt. %, preferably 0 to about 45 wt. %, more preferably 0 to about 40 wt. %, and even more preferably about 0 to 30 wt. %, based on the solids weight of the juice.

In the drying step, water is removed from the pomegranate juice obtained in the saccharide removal step or the pomegranate juice obtained in the drying aid addition step. In the drying step, the juice can be dried, for example, by vacuum concentration drying, lyophilization, spray drying, a crystal transformation process, a supercritical CO₂ process, etc., without causing a substantial impairment of the estrogen activity. Vacuum concentration drying, lyophilization and spray drying are preferable. In vacuum concentration drying, juice is concentrated and dried under reduced pressure using an evaporator or the like to provide a juice powder. In lyophilization, frozen fruit juice is dried in vacuo to provide a juice powder. In spray drying, fruit juice is atomized using a nozzle or a high speed rotary disk and the juice is instantly dried by continuously contacting juice with hot air. In other drying methods, juice can be pulverized according to conventional methods.

Pomegranate juice powder obtained by the process of producing a pomegranate juice powder of the invention contains no or a low percentage of drying aid and has an estrogen-like activity.

The food of the invention is characterized by containing the pomegranate juice powder of the invention. The form of the food of the invention is not particularly limited and examples thereof include foods, beverages and other comestibles. Specific examples include powders, supplements (pills, granules, fine granules, tablets, chewable tablets, capsules), candies, jellies, biscuits, cakes, breads, noodles and like solid or semi-solid foods; vegetable juices, vegetable/fruit blended beverages, mixed fruit/vegetable juices, and like liquid beverages; and sauces (Japanese tare), dressings, Worcester sauces, soy sauces, and like seasonings.

By incorporating the pomegranate juice powder of the invention, a food can be provided with estrogen-like activity and can be formed into a functional food, health food, food for specified health use, hospital food or the like. Preferable forms are powders, supplements (pills, granules, fine granules, tablets, chewable tablets, capsules) and like solid foods, health foods, functional foods and foods for specified health use. Such foods can be prepared according to conventional methods depending on the form of the food.

The pomegranate juice powder content of the food of the invention can be suitably selected according to the form of the food, etc. The powder content is usually about 0.1 to about 90 wt. %, and preferably about 1 to about 90 wt. %.

When the food of the invention is ingested to expect and take advantage of the estrogen-like activity, it is usually ingested in such an amount that the daily intake of the pomegranate juice powder of the invention for an adult (body weight: 50 kg) is about 0.1 to about 100 g, and preferably about 0.2 to about 10 g.

The feed of the invention is characterized by containing the pomegranate juice powder of the invention. Examples of feeds include feeds for livestock, pets and the like. The feed of the invention can be produced by adding the pomegranate juice powder of the invention to a conventional feed. Intake of the feed of the invention may be the same as the above-mentioned human intake. That is, the intake is calculated by converting the amount of human intake to a mammalian intake amount per kg body weight of the mammal to be fed.

The pharmaceutical composition of the invention is characterized by containing an effective amount of the pomegranate juice powder of the invention and may further contain pharmaceutically acceptable carrier(s). The pomegranate juice powder content as an effective component of the pharmaceutical composition may be selected as desired. The powder content is usually about 0.1 to about 90 wt. %, and preferably about 1 to about 90 wt. %. This content may be appropriately varied according to the form of the pharmaceutical composition, the age, sex and condition of the subject, etc.

The dosage unit form of the pharmaceutical composition of the invention is not particularly limited and can be selected according to the preventive or therapeutic purpose. Specific examples of dosage forms include injections, suppositories, ophthalmic solutions, ointments, aerosols and like parenteral preparations; and pills, tablets, coated tablets, powders, granules, capsules, liquids, suspensions, emulsions and like oral preparations. Preferable dosage forms are oral preparations, and pills, tablets and granules are particularly preferable. Such dosage forms can be prepared by pharmaceutical preparation processes commonly known in this field.

Examples of usable carriers include various substances used in usual pharmaceutical preparations, such as excipients, binders, disintegrators, disintegration inhibitors, absorption promoters, humectants, adsorbents, lubricants, colorants, corrigents, odor masking agents, surfactants and the like. Examples thereof include lactose, sucrose, sodium chloride, glucose, urea, starch, calcium carbonate, kaoline, crystalline cellulose, silicic acid, methylcellulose, glycerol, sodium arginate, gum arabic and like excipients; simple syrup, glucose solutions, starch solutions, gelatin solutions, polyvinyl alcohol, polyvinyl ether, polyvinylpyrrolidone, carboxymethylcellulose, shellac, methylcellulose, ethylcellulose, water, ethanol, potassium phosphate and like binders; dried starch, sodium alginate, powdered agar, powdered laminaran, sodium hydrogencarbonate, calcium carbonate, polyoxyethylene sorbitan fatty acid esters, sodium lauryl sulfate, stearic acid monoglycerides, starch, lactose and like disintegrators; sucrose, stearic acid, cocoa butter, hydrogenated oils and like disintegration inhibitors; quaternary ammonium bases, sodium lauryl sulfate and like absorption enhancers; glycerol, starch and like humectants; starch, lactose, kaolin, bentonite, colloidal silicic acid and like adsorbents; purified talc, stearic acid salts, powdered boric acid, polyethylene glycol and like lubricants; etc. Furthermore, pills and tablets may optionally be provided with a standard coating to form sugar-coated tablets, gelatin-coated tablets, enteric-coated tablets, film-coated tablets, double-layer tablets, multi-layer tablets and the like.

In preparing pills, glucose, lactose, starch, cacao butter, hydrogenated vegetable oils, kaolin, talc and like excipients; powdered gum arabic, powdered tragacanth, gelatin and like binders; laminaran, agar and like disintegants; etc., can be used as carriers.

Capsules can be prepared by mixing the active ingredient with one or more of the aforementioned various carriers and filling hard gelatin capsules, soft capsules or like known capsules with the mixture.

In preparing suppositories, polyethylene glycol, cacao butter, lanolin, higher alcohols, esters of higher alcohols, gelatin, semisynthetic glycerides, Witepsol (registered trademark, Dynamite Nobel Inc.), etc., can be used as carriers.

In preparing injections, water, ethyl alcohol, macrogol, propylene glycol, ethoxylated isostearyl alcohol, polyoxylated isostearyl alcohol, polyoxyethylene sorbitan fatty acid esters and like diluents; sodium citrate, sodium acetate, sodium phosphate and like pH adjusters; dipotassium phosphate, trisodium phosphate, sodium hydrogen phosphate, sodium citrate and like buffers; sodium pyrosulfite, EDTA, thioglycolic acid, thiolactic acid and like stabilizers; and mannitol, inositol, maltose, sucrose, lactose and like saccharides for binders in freeze-drying; etc., can be used as carriers. Sodium chloride, glucose and glycerol may be used in the pharmaceutical preparation in amounts sufficient to prepare an isotonic solution. Moreover, standard auxiliary dissolvents, soothing agents, topical anesthetics, etc., may be used. Subcutaneous, intramuscular and intravenous injections can be prepared according to conventional methods by adding such carriers.

Liquid preparations may take the form of water-based or oil-based suspensions, solutions, syrups, or elixirs, and can be prepared according to conventional methods using commonly-used additives.

In preparing the pharmaceutical preparation in a form of ointments such as pastes, creams, and gels, for example, white petrolatum, paraffin, glycerol, cellulose derivatives, polyethylene glycol, silicon, bentonite, etc., can be used as diluents.

The amount of pomegranate juice powder, which is an active ingredient of the pharmaceutical composition of the invention, varies according to the dosage form, route of administration, dosing schedule, etc., and is not particularly limited, and hence can be suitably selected. It is usually preferable that the active ingredient account for about 0.1 to about 90 wt. %, and preferably about 1 to about 90 wt. %, of the composition.

The method for administration of the pharmaceutical preparation of the invention is not limited and can be selected according to the form of the preparation, the age, sex and other characteristics of the patient, the severity of the disease, etc. The pharmaceutical preparation may be administered enterally, orally, rectally, intraorally, intraarterially, intravenously, transdermally, or in a like manner. For example, tablets, pills, solutions, suspensions, emulsions, granules, capsules, and the like are administered orally; injections are administered intraarterially or intravenously; suppositories are administered intrarectally; and ointments are applied to the skin, the mucous membranes of the mouth, etc.

The dosage of the pharmaceutical composition of the invention can be suitably selected according to the application, the age and sex of the patient, the degree of the disease, and other factors. The composition is administered in such an amount that the daily intake of the pomegranate juice powder of the invention for an adult (body weight: 50 kg) is usually about 0.1 to about 100 g, and preferably about 0.2 to about 10 g.

The pharmaceutical composition of the invention contains as an active ingredient a pomegranate juice powder having estrogen-like activity. Therefore, the composition has the effect of improving female hormone secretion. Furthermore, cholesterol reduction, antiaging effects by lipid oxidation suppression, osteoporosis prevention and treatment and like effects can also be expected from the results of the Examples shown below. Moreover, abnormal thyroid functional enhancement, which is a side effect specific to estrogen-like substances, is not observed.

The diseases to be prevented or treated by the pharmaceutical composition of the invention are those associated with female hormone reduction. Examples thereof include hot flash, poor blood circulation, palpitation, dizziness, depression, insomnia, headache, osteoporosis, hyperidrosis, osteopenia, hyperlipidemia, obesity, arteriosclerosis and the like. Osteoporosis, hyperlipidemia, obesity and arteriosclerosis are preferable target diseases. Subjects to whom the pharmaceutical composition of the invention is administered are not particularly limited. Persons with reduced female hormones and persons with diseases caused by female hormone reduction are preferable targets. Specific examples thereof are menopausal women, women before or after menopause, and persons with hormone balance disorders. Persons with hormone balance disorders include persons with reduced female hormones caused by dietary restrictions such as diets, stresses and other factors.

Since pomegranate juice powders obtained by conventional methods contain a large amount of drying aids such as dextrin, lactose and the like, such powders have a high caloric content and a low estrogen-like activity per unit weight. In contrast, since the pomegranate juice powder of the invention contains substantially no saccharides, the powder has a low caloric content and a high estrogen-like activity per unit weight. Therefore, since the powder of the invention can be added to foods in smaller amounts than conventional powders, the kinds and amounts of other additives added to the food can be more freely selected.

Furthermore, the process of producing the pomegranate juice powder of the invention can obviate the necessity of using drying aids that cause a high caloric content or can reduce the amount of drying aid used, without impairing the estrogen-like activity.

As with the pomegranate juice powder of the invention, the food of the invention has a low calorie content and a high estrogen-like activity per unit weight.

### BEST MODE FOR CARRYING OUT THE INVENTION

Examples and Comparative Examples are given below to illustrate the invention in more detail, but the scope of the invention is not limited thereby.

### EXAMPLE 1

1000 ml of a concentrated pomegranate juice (Brix 65°) was dialyzed with a dialysis membrane (molecular weight cut-off: 12000) and the fluid on the dialysis membrane was concentrated using an evaporator. The concentrate was lyophilized at −40° C. for 1 hour, followed by drying while increasing the temperature to 20° C. overnight) to give 9.26 g of a pomegranate lyophilized powder (the presence of glucose and fructose was not detected: (detection limit: 0.3 wt. %), caloric content: 50 kcal. or less). The saccharide content was determined by an enzymatic process using “F kit glucose/fructose” manufactured by Boehringer Mannheim.

9.26 g of this powder was dissolved in water to make 1000 ml of a sample juice from the powder. The sample juice and an unmarked ligand (17β estradiol) were competitively reacted with estrogen receptor α. After the reaction, the amount of free ligand in the mixture was determined by measuring the absorbance at 450 nm according to an enzymatic immunoassay using an anti-estradiol monoclonal antibody. The amount of ligand bound to the receptor was calculated from the amount of free ligand to determine the estrogen receptor binding inhibitory activity (Ligand Screening System—Estrogen Receptor α—, product of Toyobo Co., Ltd.). The inhibitory activity of 300 nM of DES (80.5 ng/ml) used as a positive control was determined in the same manner as above. Table 1 shows the inhibitory activity relative to that of the positive control defined as 100%.

### EXAMPLE 2

The same procedure as in Example 1 was followed except that the membrane used in Example 1 was replaced by a dialysis membrane with a molecular weight cut-off of 3500 to give 4.25 g of a pomegranate lyophilized powder (the presence of glucose and fructose was not detected: (detection limit: 0.3 wt. %), caloric content: 50 kcal. or less). The estrogen receptor binding inhibitory activity of this powder was determined in a manner similar to Example 1. Table 1 shows the results.

### EXAMPLE 3

The same procedure as in Example 1 was followed except that the membrane used in Example 1 was replaced by a dialysis membrane with a molecular weight cut-off of 8000 to give 5.00 g of a pomegranate lyophilized powder (the presence of glucose and fructose was not detected: (detection limit: 0.3 wt. %), caloric content: 50 kcal. or less). The estrogen receptor binding inhibitory activity of this powder was determined in a manner similar to Example 1. Table 1 shows the results.

### EXAMPLE 4

The same procedure as in Example 1 was followed except that the membrane used in Example 1 was replaced by a dialysis membrane with a molecular weight cut-off of 2000 to give 8.84 g of a pomegranate lyophilized powder (the presence of glucose and fructose was not detected: (detection limit: 0.3 wt. %), caloric content: 50 kcal. or less). The estrogen receptor binding inhibitory activity of this powder was determined in a manner similar to Example 1. Table 1 shows the results.

### COMPARATIVE EXAMPLE 1

A pomegranate juice powder was prepared using a drying aid without removing saccharides from the pomegranate juice. More specifically, 980 g of dextrin (the minimum amount necessary for pulverization) was added to 1000 ml of a 5 times concentrated pomegranate juice, followed by spray drying to give 1543 g of a pomegranate juice powder (caloric content: 5850 kcal.). This powder was diluted with water to make 1000 ml of a sample juice from the powder. Because the juice from the powder was considered to have the same inhibitory activity as the 5 times concentrated juice, the estrogen receptor binding inhibitory activity of this juice was assumed to be 99%. Table 1 shows the caloric content and inhibitory activity.

TABLE 1

| | | | | | | 5 times |
|---|---|---|---|---|---|---|
| | | | | | | concentrated |
| | | | | | Comp. | juice |
| | Example 1 | Example 2 | Example 3 | Example 4 | Ex. 1 | (1000 ml) |
| Molecular | 12000 | 3500 | 8000 | 2000 | — | — |
| weight cut-off | | | | | | |
| Proportion | 95% or | 95% or | 95% or | 95% or | 0% | 0% |
| of glucose | more | more | more | more | | |
| and fructose | | | | | | |
| removed | | | | | | |
| Caloric | 50 or | 50 or | 50 or | 50 or | 5850 | 2360 |
| content | less | less | less | less | | |
| (kcal.) | | | | | | |
| Inhibitory | 79% | 96% | 94% | 96% | 99% | 99% |
| activity | | | | | | |

It was confirmed that the aqueous solutions of pomegranate juice powders obtained in Examples 1 to 4 (juices reconstituted from powders) had substantially the same inhibitory activity as the 5 times concentrated pomegranate juice but that their caloric contents were 1/50 or less that of the 5 times concentrated pomegranate juice and 1/100 or less that of the powder of Comparative Example 1. In contrast, the pomegranate juice powder of Comparative Example 1 prepared using a drying aid without removing saccharides from the pomegranate juice had the same estrogen-like activity as the 5 times concentrated pomegranate juice but its caloric content was at least twice that of the 5 times concentrated pomegranate juice.

To compare estrogen-like activity strengths, each sample was prepared at several concentrations and their 50% inhibitory concentrations (IC₅₀) were determined in a manner similar to the above (Ligand Screening System—Estrogen Receptor α—, product of Toyobo Co., Ltd.).

For example, the IC₅₀ of the powder of Example 1 was 900 ng/ml, and that of DES was 2.5 ng/ml. Accordingly, the estrogen-like activity of this powder was 1/360 that of DES. Since the activity of 1 g of this powder is equivalent to the activity of 2800 μg of DES, the activity was calculated as 2800 U.

Table 2 shows the results of a comparison of the activities of Example 1, Comparative Example 1, and 5 times concentrated fruit juice per unit weight.

TABLE 2

| | | 5 times | | |
|---|---|---|---|---|
| | | concentrated | | Comparative |
| | DES | juice | Example 1 | Example 1 |
| IC₅₀ | 0.0025 μg/ml | 80 μg/ml | 0.9 μm/ml | 80 μm/ml |
| Activity | — | 48 U/g | — | 48 U/g |
| per gram of | | | | |
| glucose and | | | | |
| fructose | | | | |
| Activity | 10⁶ U/g | — | 2800 U/g | 24 U/g |
| per gram of | | | | |
| the powder | | | | |

Table 3 below shows the caloric contents of 0.3 g of the powder of Example 1 and 43.5 g of the powder of Comparative Example 1. These are the amounts of powders capable of providing the same estrogen-like activity as 30 ml of 5 times concentrate pomegranate juice (i.e., the daily intake expected to exhibit estrogen-like activity effects).

TABLE 3

| | 5 times | | |
|---|---|---|---|
| | concentrated | | Comparative |
| | juice | Example 1 | Example 1 |
| Activity | 940 U | 940 U | 940 U |
| Amount | 30 ml | 0.3 g | 43.5 g |
| Calorie | 70.8 kcal. | 1.3 kcal. | 165 kcal. |
| | | or less | |

To obtain the same level of estrogen-like activity as the 5 times concentrated juice, 0.3 g of the pomegranate juice powder of Example 1 is necessary and its caloric content is 1.3 kcal. In contrast, 43.5 g of the pomegranate juice powder of Comparative Example 1 is needed and its caloric content is 165 kcal. That is, when a powder is ingested to provide an estrogen-like activity corresponding to that of 30 ml of the 5 times concentrated juice, 43.5 g (165 kcal.) of the powder of Comparative Example 1 needs to be ingested, thus resulting in a high calorie intake. In contrast, the required intake of the powder of Example 1 is 0.3 g (1.3 kcal.) or less, so that even a small amount of the powder provides a potent estrogen-like activity at a low caloric content that is easy to form into tablets, capsules, etc. and is advantageous as a portable supplement.

### EXAMPLE 5

A 5 times concentrated pomegranate juice (saccharide concentration: 65 wt. %) was diluted 20 times with water and a yeast (Saccharomyces cereviciae) was inoculated therein and incubated at 30° C. for 5 days to give a pomegranate juice with a saccharide concentration of 1 wt. % (about 4 kcal./100 ml). This juice was atomized using a nozzle or a high speed rotary disk and continuously contacted with hot air to dry instantly by spray drying, thereby evaporating the alcohol, thus giving a pomegranate juice powder. The powder had a saccharide concentration of 1 wt. % and its caloric content was 4 kcal./100 ml.

### FORMULATION EXAMPLE 1

A pomegranate juice powder prepared in the same manner as Example 3 was compressed into 150 mg tablets. Since 1 tablet contained 150 mg of the pomegranate juice powder, an intake of 1 tablet per day is expected to provide estrogen-like activity effects.

### FORMULATION EXAMPLE 2

A pomegranate juice powder prepared in the same manner as in Example 3, lactose, and crystalline cellulose were mixed at a weight ratio of 1:1:1, granulated and compressed into 150 mg tablets. Since 1 tablet contained 50 mg of the pomegranate juice powder, an intake of 3 tablets per day is expected to provide estrogen-like activity effects. The caloric content per tablet is about 2.4 kcal.

### FORMULATION EXAMPLE 3

A pomegranate juice powder prepared in the same manner as in Example 1 was mixed with dextrin at a weight ratio of 1:1 and granulated in a fluidized bed to give granules. Since 1 g of the granules contained 500 mg of the pomegranate juice powder, an intake of 0.5 g per day is expected to provide estrogen-like activity effects.

### FORMULATION EXAMPLE 4

A pomegranate juice powder prepared in the same manner as in Example 1 was mixed with dextrin at a weight ratio of 1:2 and placed into commercially available capsules to provide capsules with a powder plus dextrin content of 250 mg per capsule. Since 1 capsule contains 73 mg of the pomegranate juice powder, a daily intake of 6 capsules is expected to provide estrogen-like activity effects corresponding to those achieved by 30 ml of concentrated pomegranate juice.

### TEST EXAMPLE

Subjects were instructed to take capsules of Formulation Example 4 and their cholesterol, sex hormone, bone marker, thyroid function marker, anti-oxidation marker, and anti-aging marker values, etc. were measured before intake of the capsules and after 8 weeks of intake.

More specifically, 11 healthy women aged 18 or more were used as subjects. Before the test, the blood, urea and saliva of fasted subjects were sampled. With respect to the saliva, the subjects were instructed to sample saliva with a sampling kit every 6 hours for a total of 24 hours. Using these samples, measurements were made with respect to the items shown below.

Each subject received 336 capsules and was instructed to take 2 capsules 3 times per day with a meal. The intake period was 8 weeks (56 days). After the completion of the intake, the blood, urea and saliva were sampled in the same manner as sampled before the test and collected and measured for each test item.

(1) Cholesterol

Table 4 shows the results for serum total cholesterol, LDL cholesterol and HDL cholesterol. The table shows the mean values. Ratios were calculated by the following formula:
value measured after 8 weeks of intake/value measured before intake×100.

Compared to the values before testing, HDL cholesterol increased in 9 out of 11 subjects and the total cholesterol/HDL ratio also decreased in 9 out of 11 subjects. Moreover, LDL cholesterol decreased in 7 subjects. A cholesterol balance improving effect was confirmed. Therefore, the intake of the pomegranate juice powder of the invention can be expected to be effective in suppressing hyperlipidemia.

TABLE 4

| | | After 8 | |
|---|---|---|---|
| | Before | weeks of | After/Before |
| | intake | intake | (%) |
| Total cholesterol (mg/dl) | 174.5 | 190.2 | 109.4 |
| LDL (mg/dl) | 123.1 | 115.0 | 93.8 |
| HDL (mg/dl) | 52.3 | 69.2 | 134.3 |
| Total cholesterol/HDL | 3.5 | 2.9 | 83.4 |

(2) Sex Hormone

Table 5 shows the results mean values for serum estradiol, serum testosterone, saliva estradiol and saliva testosterone. Ratios were calculated by the following formula:
value measured after 8 weeks of intake/value measured before intake×100.

Serum estradiol and serum testosterone increased in 10 out of 11 subjects, and saliva estradiol and saliva testosterone increased in 8 subjects. The pomegranate juice powder of the invention is considered to be effective in improving various symptoms caused by female hormone reduction.

TABLE 5

| | | After 8 | |
|---|---|---|---|
| | Before | weeks of | After/Before |
| | intake | intake | (%) |
| Serum estradiol (pg/ml) | 80.5 | 126.0 | 157.1 |
| Saliva estradiol (pg/ml) | 2.5 | 2.9 | 113.6 |
| Serum testosterone (ng/dl) | 39.0 | 40.7 | 109.6 |
| Saliva testosterone (ng/dl) | 37.1 | 37.0 | 101.6 |

(3) Bone Markers

Table 6 shows the results mean values for calcitonin, ostecalcin and bone-specific alkaline phosphatase. Percentages were calculated by the following formula:
ratios measured after 8 weeks of intake/value measured before intake×100

The values decreased for every item in all the subjects. The pomegranate juice powder of the invention is considered to be effective for preventing and improving osteoporosis.

TABLE 6

| | | After 8 | |
|---|---|---|---|
| | Before | weeks of | After/Before |
| | intake | intake | (%) |
| Calcitonin (pg/ml) | 3.8 | 2.9 | 77.6 |
| Osteocalcin (ng/ml) | 5.3 | 3.7 | 71.8 |
| Bone-specific alkaline | 32.1 | 23.6 | 75.3 |
| phosphatase (U/I) | | | |

(4) Thyroid Functions

Table 6 shows the results mean values for serum T₃ uptake ratios, total thyroxine (T₄), free thyroxine, and thyrotropic hormone. After 8 weeks of intake, no abnormal values were observed for any item. The administration of estrogen is known to result in abnormally low T₃ uptake ratios and abnormally high thyrotropic hormone levels. In contrast, no adverse effects on thyroid functions were observed with the pomegranate juice powder of the invention.

TABLE 7

| | | | After 8 |
|---|---|---|---|
| | Normal | Before | weeks of |
| | range | intake | intake |
| T₃ uptake ratio (%) | 32-45 | 38.0 | 36.7 |
| Total thyroxine (T₄) (μ/dl) | 5-12 | 9.1 | 7.8 |
| Isolation thyroxine (ng/dl) | 5-11 | 10.0 | 8.3 |
| Thyrotropic hormone (ng/dl) | 0.35-5 | 1.4 | 1.2 |

(5) Anti-Oxidative Effect and Anti-Aging Effect

Table 8 shows the results mean values for urine lipid peroxide, which is an anti-oxidation marker. After 8 weeks of intake, urine lipid peroxide levels decreased in 8 out of 11 subjects, indicating a suppression of lipid oxidation. Table 8 also shows the results (mean values) for serum somatomedin, which is an anti-aging marker. After 8 weeks of intake, somatomedin levels increased in 8 out of 11 subjects. The above results suggest that the pomegranate juice powder of the invention has an anti-aging action. Ratios were calculated by the following formula:
value measured after 8 weeks of intake/value measured before intake×100.

TABLE 8

| | Before | After 8 weeks | After/Before |
|---|---|---|---|
| | intake | of intake | (%) |
| Urine lipid peroxide | 5.3 | 3.5 | 640 |
| (nmol/ml) | | | |
| Somatomedin (ng/ml) | 204.3 | 224.8 | 109.7 |

### INDUSTRIAL APPLICABILITY

According to the present invention, a pomegranate juice with enhanced estrogen-like activity per unit amount, a pomegranate juice powder, a process for producing the powder, and foods, feeds, pharmaceutical compositions and the like containing the powder can be provided.

1. A fruit juice derived from pomegranate juice, comprising substantially no glucose and no fructose.

2. A fruit juice derived from a pomegranate juice, the fruit juice containing not more than 50% of the glucose and fructose of the pomegranate juice and having about 50% or more of the estrogen-like activity of the pomegranate juice.

3. A fruit juice derived from pomegranate juice and having an estrogen-like activity of about 96 U or more per gram of glucose and fructose contained in the fruit juice.

4. A pomegranate juice powder having an estrogen-like activity of about 40 U or more per gram of the powder.

5. The powder according to claim 4 having an estrogen-like activity of about 40 to about 20,000 U per gram of the powder.

6. The powder according to claim 4 further comprising a drying aid.

7. A pomegranate juice powder containing glucose and/or fructose and having an estrogen-like activity of about 80 U or more per gram of the glucose and fructose contained therein.

8. The powder according to claim 7 further comprising a drying aid.

9. A process for producing a pomegranate juice powder comprising a saccharide removal step of removing glucose and fructose from a pomegranate juice, and a drying step of drying the juice obtained by the saccharide removal step.

10. The process according to claim 9 wherein the succharide removal step is performed by a membrane treatment using at least one membrane with a molecular weight cut-off of about 200 to about 100,000 selected from the group consisting of dialysis membranes, ultrafiltration membranes, nano filters, and reverse osmotic membranes, or by chromatography using a packing with a molecular weight cut-off of about 200 to about 100,000 to obtain a higher molecular weight fraction.

11. The process according to claim 9 wherein the saccharide removal step is performed by a microbiological method comprising treating the juice with a microorganism which utilizes glucose and/or fructose to produce an alcohol, or by an enzymatic method comprising treating the juice with an enzyme that uses glucose and/or fructose as substrate(s).

12. The process according to claim 10 which comprises an additional step, between the saccharide removal step and the drying step, of adding a drying aid to the juice from which saccharide(s) have been removed.

13. A pomegranate juice powder obtainable by the method of claim 9 and having estrogen-like activity.

14. A food comprising the pomegranate juice powder of claim 4.

15. A feed comprising the pomegranate juice powder of claim 4.

16. A pharmaceutical composition comprising an effective amount of the pomegranate juice powder of claim 4.

17. The pharmaceutical composition according to claim 16 which is a preventive or therapeutic agent for a disease associated with female hormone reduction.

18. A method of preventing or treating a disease associated with a female hormone, comprising orally administering an effective amount of the pomegranate juice powder of claim 4 to a subject.

19. The powder according to claim 5 further comprising a drying aid.

20. The process according to claim 12 which comprises an additional step, between the saccharide removal step and the drying step, of adding a drying aid to the juice from which saccharide(s) have been removed.
